(19) United States
(12) Patent Application Publication
(10) Pub. No.: US 2006/0141021 A1
(43) Pub. Date: Jun. 29, 2006

(54) Polymeric microspheres and method for preparing the same

(75) Inventors: Ae-June Wang, Hsinchu (TW); Yi-Fong Lin, Jhonghe City (TW); Chi-Heng Jian, Jiaosi Township (TW); Shin-Jr Liu, Niaosong Township (TW)

Correspondence Address: THOMAS, KAYDEN, HORSTEMEYER & RISLEY, LLP, 100 GALLERIA PARKWAY, NW, STE 1750, ATLANTA, GA, 30339-5948, US

(73) Assignee: Industrial Technology Research

(21) Appl. No.: 11/223,063

(22) Filed: Sep. 9, 2005

Related U.S. Application Data
(63) Continuation-in-part of application No. 11/024,904, filed on Dec. 29, 2004.

(51) Int. Cl. A61K 9/127

(52) U.S. Cl. 424/450

(57) ABSTRACT

A polymeric microsphere. The polymeric microsphere comprises a first polymer, a layer formed on the surface of the first polymer, and a second polymer formed on the layer. The invention also provides a method for preparing the polymeric microphere by an aqueous-two-phase emulsion process.

This application is a continuation-in-part of application Ser. No. 11/024,904 filed on Dec. 29, 2004, now pending.

### BACKGROUND

The present invention relates to a polymeric microsphere, and more particularly to a method for preparing a polymeric microsphere by an aqueous-two-phase emulsion process using two miscible polymer solutions.

Polymeric microsphere preparation can be classified into spraying and emulsion methods. The spraying method can be seen in U.S. Pat. No. 6,238,705. A polymer with cross-linking properties, such as sodium alginate, is sprayed from a nozzle into an ionic cross-linking agent with a +2 charge. Chitosan is then adsorbed on the surface of the microspheres. This method does not use an organic solvent or a surfactant in the process. The recovery yield, however, is poor, losing about 20-30% due to nozzle spray wastages.

Regarding to the emulsion methods, an oil/water emulsion method for preparing such polymeric microspheres is first provided, such as an oil-in-water or water-in-oil emulsion method. For example, in EP 0480729, a lipophilic drug, such as a steroid drug or an anticancer drug, is dissolved in an oil phase and then emulsified into an aqueous phase (a polysaccharide polymer or a mixture thereof), thus forming oil-in-water polymeric microspheres. This method, however, requires addition of an organic solvent or surfactant. Also, the organic solvent must be removed at high temperature during the preparation process. Biological drugs, such as peptides and proteins, are less stable than small molecule drugs in this method due to addition of organic solvents or surfactants, causing denaturation and loss of their activities.

In order to avoid requirement of an organic solvent or a surfactant to achieve high recovery, an aqueous-two-phase method has been applied to prepare polymeric microspheres. In 1995, Gehrke provided a dextran/PEG aqueous-two-phase system composed of two immiscible polymers (Proceed. Intern. Symp. Control Rel. Bioact. Material., 22, 145-146).

EP 0213303 discloses many aqueous-two-phase systems with polymer compositions of dextran-alginate/PEG, carboxymethylcellulose/PEG, and starch/PEG. Each system is, again, composed of two immiscible polymers.

In U.S. Pat. No. 5,204,108, Illum discloses an aqueous-two-phase system, such as starch/PEG, albumin/PEG, or gelatin/PEG, to encapsulate insulin. This system is composed of two immiscible polymers. In addition, glutaldehyde is used as a microsphere cross-linking agent.

Lamberti provides a dextran-alginate/PEG system disclosed in U.S. Pat. No. 5,827,707, which includes two immiscible polymers. Alginate is cross-linked for preparation as an implantable microcapsule.

In 2001, Hennink (U.S. Pat. No. 6,303,148) disclosed a controlled release aqueous-two-phase system, such as dextran-GMA/PEG and dextran-lactHEMA/PEG. The modified dextran-GMA can be cross-linked to form microspheres without alginate. This system can be used to encapsulate protein drugs or genes. At least 80 wt % of the microspheres had a particle size between 100 nm and 100 μm.

In the described literature and patents for the preparation of polymeric microspheres, the spraying method has poor recovery yield, and the oil/water emulsion method easily denatures the encapsulated biological drugs during the process. Additionally, requirement for two immiscible polymers also limits the selectivity of polymers.

### SUMMARY

An object of the present invention is to solve the above-mentioned problems and to provide an aqueous-two-phase emulsion method for preparing polymeric microspheres. The present invention requires no organic solvents or surfactants. Therefore, encapsulated biological drugs remain activity during the preparation process. Also, recovery yield and encapsulation efficiency of the drugs are increased.

Thus, a polymeric microsphere is provided. The polymeric microsphere comprises a first polymer, a layer formed on the surface of the first polymer, and a second polymer formed on the layer.

An aqueous-two-phase emulsion method for preparing polymeric microspheres is provided, comprising the following steps. A first polymer aqueous solution is provided comprising functional groups capable of cross-linking. A second polymer aqueous solution is provided, miscible with the first polymer aqueous solution. The first and second polymer aqueous solutions are mixed to form a mixture. Finally, a crosslinking agent is added to form a microsphere.

The polymeric microspheres prepared by the aqueous-two-phase process can encapsulate a drug. Thus, the present invention also provides a method for preparing polymeric microspheres encapsulated with drugs, comprising the following steps. A first polymer aqueous solution is provided comprising functional groups capable of cross-linking. A second polymer aqueous solution is provided, miscible with the first polymer aqueous solution. Drugs and the first polymer aqueous solution are mixed to form a drug aqueous solution. The drug aqueous solution and the second polymer aqueous solution are mixed to form a mixture. Finally, a crosslinking agent is added to form a microsphere.

The present invention further provides a method for preparing polymeric microspheres containing a plurality of liposomes, comprising the following steps. A first polymer aqueous solution is provided comprising functional groups capable of cross-linking. A second polymer aqueous solution is provided, miscible with the first polymer aqueous solution. A liposome solution and the first polymer aqueous solution are mixed to form a solution. The solution and the second polymer aqueous solution are mixed to form a mixture. Finally, a crosslinking agent is added to form a microsphere.

A detailed description is given in the following embodiments with reference to the accompanying drawings.

### BRIEF DESCRIPTION OF THE DRAWINGS

Embodiments of the invention can be more fully understood by reading the subsequent detailed description and examples with references made to the accompanying drawings, wherein:

FIG. 1 shows surface cross-linking of the polymer by hydrogen bonding.

FIG. 2 shows ionic cross-linking of the polymer.

FIG. 3 is a schematic diagram of the aqueous-two-phase process for preparing polymeric microspheres in continuous homogenization.

### DETAILED DESCRIPTION

The invention provides a polymeric microphere comprising a first polymer, a layer formed on the surface of the first polymer, and a second polymer formed on the layer.

The microsphere may encapsulate a plurality of liposomes, drugs, or liposomes encapsulated with drugs.

The invention also provides an aqueous-two-phase emulsion method for preparing polymeric microspheres, comprising the following steps. A first polymer aqueous solution is provided comprising functional groups capable of cross-linking. A second polymer aqueous solution is provided, miscible with the first polymer aqueous solution. The first and second polymer aqueous solutions are mixed to form a mixture. Finally, a crosslinking agent is added to form a microsphere.

The aqueous-two-phase emulsion process of the present invention uses two miscible polymer solutions. One polymer (the first polymer) has functional groups capable of cross-linking. For example, the first polymer can be a carboxylate polymer, that is, a polymer with carboxylate (COO⁻) or carboxyl (COOH) groups. Representative examples comprise alginic acid, alginate such as sodium alginate, propylene glycol alginate, carboxylmethyl cellulose, polyacrylic acid, or polyacrylate derivatives.

The other polymer (the second polymer) is not limited, provided that it is miscible with the first polymer. Representative examples of the second polymer comprise chitosan, starch, dextran, hydroxyl propyl methyl cellulose, or gelatin.

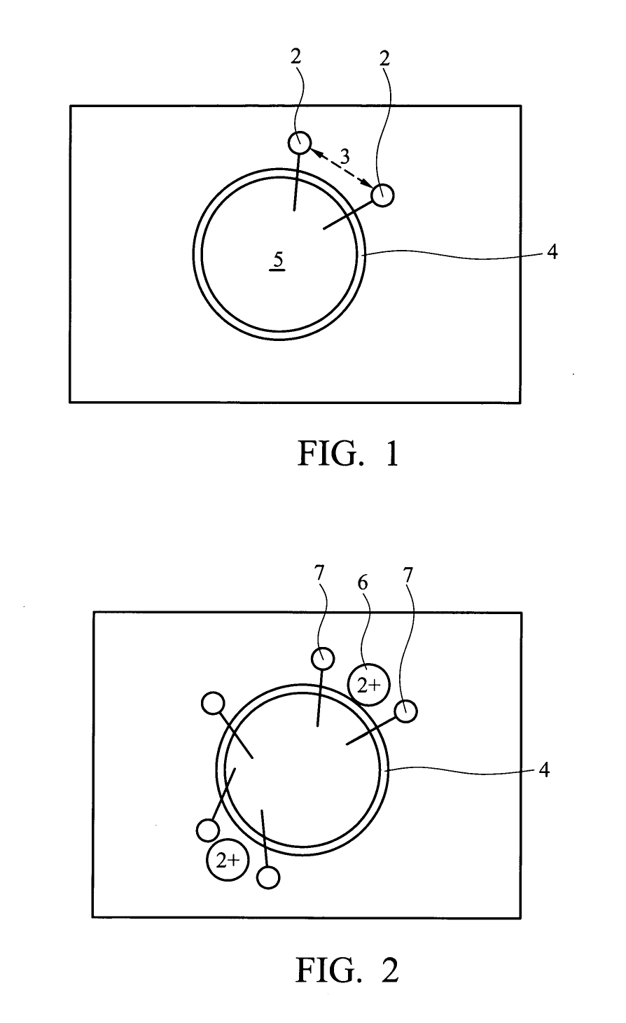

The second polymer solution 1 is adjusted to acidity. Then, the first and the second polymer aqueous solutions are mixed and stirred, for example, homogenized in a homogenizer, to form an emulsion. The first polymer aqueous solution forms a dispersed phase (comprising a plurality of microspheres) in a continuous phase of the second polymer aqueous solution. The COO⁻ or COOH groups 2 on the first polymer surface are cross-linked and form hydrogen bonds 3 therebetween. A layer 4 is formed on the surface of the first polymer 5 (carboxylate polymer), as shown in FIG. 1. The layer (protective film) formed by cross-linking of COO⁻ or COOH groups 2 prevents mutual dissolution of the inner and outer polymers.

Subsequently, in order to stabilize and enhance polymeric microsphere structures, a cross-linking agent 6, such as an ionic cross-linking agent with +2 charges, can be added to initiate the cross-linking between COO⁻ 7 and the ionic cross-linking agent, as shown in FIG. 2. The polymeric microsphere obtained from the invention has a particle size between 0.1 μm and 100 μm.

According to the invention, the second polymer is preferably adjusted to acidity, for example, pH 0.5 to 6, most preferably pH 1.5 to 5. Generally, the cross-linking agent added has almost the same pH as the second polymer aqueous solution. The pH of the first polymer aqueous solution is not limited and can be, for example, 2 to 13.

The first polymer aqueous solution has a concentration exceeding 1%, preferably 2% to 10%. The second polymer aqueous solution has a concentration exceeding 0.5%, preferably 1% to 10%.

The weight of the second polymer aqueous solution is 1.5 to 20 times, preferably 2 to 3 times, the weight of the first polymer aqueous solution.

Further, the polymers obtained from the present invention, prepared by the aqueous-two-phase emulsion process using two miscible polymer solutions, are used to encapsulate drugs, as described herein. Drugs and the first polymer aqueous solution are mixed to form a drug aqueous solution. The second polymer aqueous solution is adjusted to acidity. Then, the drug aqueous solution and the second polymer aqueous solution are mixed to form a mixture.

As mentioned above, COO⁻ or COOH groups on the first polymer surface form hydrogen bonds therebetween and are cross-linked to form a layer on the surface of the first polymer, as shown in FIG. 1. The drugs are encapsulated in the microspheres but not shown. The layer (protective film) formed by cross-linking prevents mutual dissolution of the inner and outer polymers. Further, release of the drugs to the outer phase is prevented by the layer, thus increasing encapsulation efficiency (E.E.).

Subsequently, in order to stabilize and strengthen the polymeric microsphere structures, a cross-linking agent, such as an ionic cross-linking agent with +2 charges, can be added to initiate cross-linking between COO⁻ and the ionic cross-linking agent, as shown in FIG. 2. The drug-encapsulated polymeric microsphere obtained from the present invention has a particle size between 0.1 μm and 100 μm.

According to the invention, drugs suitable for encapsulation in the polymer microsphere are not limited, and can comprise, for example, small molecule drugs, peptides, proteins or liposomes with various charges.

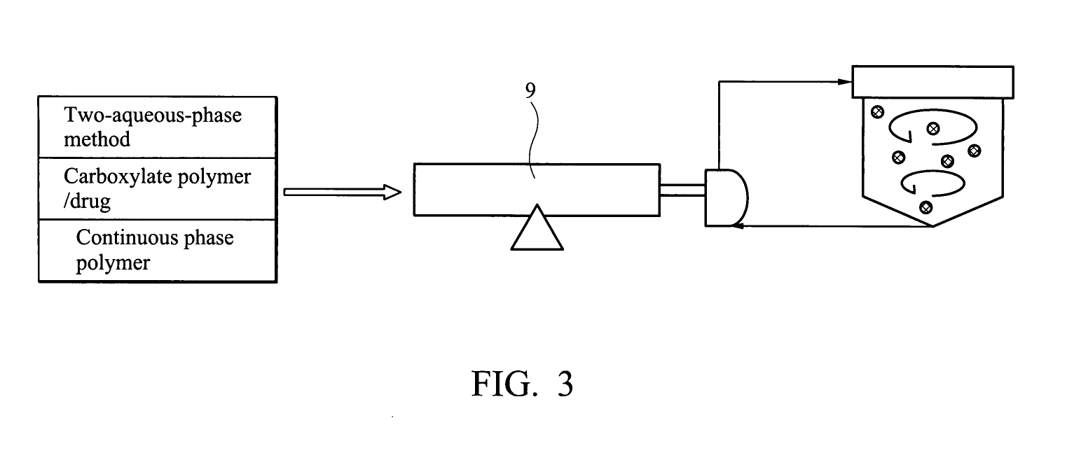

Homogenization used in the present invention can be batch homogenization or continuous homogenization. The method of the invention is suitable for a scale-up process. After the aqueous-two-phase emulsion process is performed, continuous homogenization 9 (shown in FIG. 3) is preferably used for the large scale emulsion.

The invention further provides a method for preparing polymeric microspheres encapsulating a plurality of liposomes, comprising the following steps. A first polymer aqueous solution is provided comprising functional groups capable of surface cross-linking. A second polymer aqueous solution is provided, miscible with the first polymer aqueous solution. A liposome solution and the first polymer aqueous solution are mixed to form a solution. The solution and the second polymer aqueous solution are mixed to form a mixture. Finally, a crosslinking agent is added to form a microsphere. The invention further provides a method for preparing polymeric microspheres encapsulating a plurality of liposomes encapsulated with drugs and this method is similar to the foregoing process, with the distinction therebetween merely that the latter provides a liposome solution mixed with drugs to mix with the first polymer aqueous solution.

Without intending to limit it in any manner, the invention is further illustrated by the following examples.

### EXAMPLE 1

### Preparation of Polymeric Microsphere

1 g of sodium alginate was completely dissolved to form a 10% sodium alginate aqueous solution. 2 g of chitosan was dissolved to form a 1.5% aqueous solution (pH 4.4). These two aqueous solutions were mixed and homogenized at 9500 rpm for 30 minutes to form an emulsion. 1 g of calcium chloride solution (4.5%, pH 4.4) was slowly dropped into the emulsion and stirred for 30 minutes, allowing sodium alginate to crosslink to form polymeric microspheres. The resulting microspheres were filtered off under reduced pressure. The filter cake was dispersed in water for 10 minutes (filter cake:water=1:3(w/w)), and then frozen at −20° C. for 3 hours. After complete freezing, the sample was freeze-dried for 24 hours, that is, frozen at −40° C. for 60 minutes and then dried at 4° C. until completely dry, obtaining dried polymeric microspheres.

### EXAMPLE 2

### Preparation of Polymeric Microsphere

1 g of sodium alginate was completely dissolved to form a 10% sodium alginate aqueous solution. 2 g of dextran was dissolved to form a 10% aqueous solution (pH 1.0). These two aqueous solutions were mixed and homogenized at 9500 rpm for 30 minutes to form an emulsion. 1 g of calcium chloride solution (6%, pH 1.0) was slowly dropped into the emulsion and stirred for 30 minutes, allowing sodium alginate to crosslink and form polymeric microspheres. The resultant microspheres were filtered off under reduced pressure. The filter cake was dispersed in water for 10 minutes (filter cake:water=1:3(w/w)), and then frozen at −20° C. for 3 hours. After complete freezing, the sample was freeze-dried for 24 hours, that is, frozen at −40° C. for 60 minutes and then dried at 4° C. until completely dry, obtaining dried polymeric microsphere.

### EXAMPLE 3

### Preparation of Polymeric Microsphere

1 g of Carbopol 934P (CP 934P, manufactured from BF Goodrich) was completely dissolved in 0.5N NaOH to form a 3% Carbopol aqueous solution (pH 13). 2 g of chitosan was dissolved in water to form a 2% aqueous solution (pH 2.0). These two aqueous solutions were mixed and homogenized at 9500 rpm for 30 minutes to form an emulsion. 1 g of zinc sulfate solution (6%, pH 2.0) was slowly dropped into the emulsion and stirred for 30 minutes, allowing Carbopol to crosslink and form polymeric microspheres. The resultant microspheres were filtered off under reduced pressure. The filter cake was dispersed in water for 10 minutes (filter cake:water=1:3(w/w)), and then frozen at −20° C. for 3 hours. After complete freezing, the sample was freeze-dried for 24 hours, that is, frozen at −40° C. for 60 minutes and then dried at 4° C. until completely dry, obtaining dried polymeric microspheres.

### EXAMPLE 4

### Preparation of Liposomes Encapsulated with Calcitonin

0.5 g lipid was added to a 15 mL tube. The lipid contained soybean phosphatidylcholine, TPGS, and cholesterol with a molar ratio of 20:1:1. Proper quantities of salmon calcitonin were dissolved in 0.05M citrate buffer solution (pH 4.4). 0.5 ml salmon calcitonin solution (10 mg/ml) was then added to the lipid and mixed with vortex at 200 rpm for 1 hour to form a colloid. 0.2 g colloid and 1.8 mL citrate buffer solution were then added to a 10 mL flask and hydrated at room temperature for 1 hour. A calcitonin liposome solution was prepared. The liposome solution had a calcitonin concentration of 0.56 mg/mL and an encapsulation efficiency of 86%.

### EXAMPLES 5 and 6

The procedures as described in Example 4 were again employed, except that lipid formulations and citrate buffer solution concentrations were changed. The results are shown in Table 1.

TABLE 1

| | | | | | Di- | | Inner | Hydration |
|---|---|---|---|---|---|---|---|---|
| Ex- | | | | | am- | | buffer | buffer |
| am- | | | | E.E. | eter | | (citrate, | (citrate, |
| ple | SPC | Chol. | TPGS | (%) | (nm) | PI | M) | M) |
| 4 | 20 | 1 | 1 | 86 | 339.1 | 0.13 | 0.05 | 0.10 |
| 5 | 10 | 1 | 1 | 73.2 | 238.9 | 0.30 | 0.00 | 0.10 |
| 6 | 10 | 1 | 1 | 66.8 | 257.8 | 0.28 | 0.05 | 0.05 |

In calcitonin liposome preparation, removal of drugs from inner buffer to hydration buffer is decreased during hydration due to formation of concentration gradient between liposome core and hydration buffer, increasing encapsulation efficiency.

### EXAMPLE 7

### Preparation of Liposomes Encapsulated with Calcitonin

0.5 g lipid was added to a 15 mL tube. The lipid contained soybean phosphatidylcholine, TPGS, cholesterol, and medium-chain triglyceride oil (3575oil) with a molar ratio of 10:1:2:1.16. Proper quantities of salmon calcitonin were dissolved in 0.05M citrate buffer solution (pH 4.4). 0.5 mL salmon calcitonin solution (10 mg/mL) was then added to the lipid and mixed with vortex at 200 rpm for 1 hour to form a colloid. 0.2 g colloid and 1.8 mL citrate buffer solution were then added to a 10 ml flask and hydrated at room temperature for 1 hour. A calcitonin liposome solution was prepared. The liposome solution had a calcitonin concentration of 0.7 mg/mL and an encapsulation efficiency of 80.7%.

### EXAMPLE 8

The procedures as described in Example 7 were again employed, except that lipid formulation was changed. The results are shown in Table 2.

TABLE 2

| | | | | | | | Diam- | |
|---|---|---|---|---|---|---|---|---|
| Exam- | | | | | 3575 | E.E. | eter | |
| ple | SPC | Chol. | TPGS | Brij 35 | oil | (%) | (nm) | PI |
| 7 | 10 | 2 | 1 | | 1.16 | 80.7 | 259.5 | 0.18 |
| 8 | 10 | 2 | | 1 | 1.16 | 86.5 | 251 | 0.31 |

The encapsulation efficiency of liposomes is increased (>80%) by adding 3575oil. Currently, the highest encapsulation efficiency is 47.8% disclosed in Life sciences, vol. 53, pp. 1279-1290 (1993).

### EXAMPLES 9-11

### Preparation of Liposomes Encapsulated with Calcitonin

0.5 g lipid was added to a 15 mL tube. The lipid contained soybean phosphatidylcholine, TPGS, cholesterol, and DPPG with a molar ratio of 8:1:1:2. Proper quantities of salmon calcitonin were dissolved in 0.05M citrate buffer solution (pH 4.4). 0.5 mL salmon calcitonin solution (60 mg/mL) was then added to the lipid and mixed with vortex at 200 rpm for 1 hour to form a colloid. 0.2 g colloid and 1.8 mL citrate buffer solution were then added to a 10 mL flask and hydrated at room temperature for 1 hour. A calcitonin liposome solution was prepared. The liposome solution had a calcitonin concentration of 5.77 mg/mL and an encapsulation efficiency of 100%. The results are shown in Table 3.

TABLE 3

| | | | | | | Drug | | | |
|---|---|---|---|---|---|---|---|---|---|
| | | | | | Stock | content | E.E. | Diameter | |
| Example | SPC | DPPG | Chol. | TPGS | (mg/mL) | (mg/ml) | (%) | (nm) | PI |
| 9 | 8 | 2 | 1 | 1 | 60 | 5.77 | 100 | 277.3 | 0.416 |
| 10 | 8 | 2 | 1 | 1 | 30 | 3.63 | 101.9 | 249 | 0.262 |
| 11 | 8 | 2 | 1 | 1 | 60 | 5.72 | 99.4 | 261.6 | 0.29 |

The DPPG (anionic lipid) significantly increases encapsulation efficiency and drug contents.

### EXAMPLE 12

### Preparation of Polymeric Microsphere Encapsulated with Calcitonin Liposomes

1 g of sodium alginate was completely dissolved in water to form a 10% sodium alginate aqueous solution, and then mixed with the same amount of a calcitonin liposome solution. After complete dissolution, 1 g of the sodium alginate/liposome solution and 2 g of a chitosan solution (1.5%, pH 2.0) were mixed and homogenized at 9500 rpm for 30 minutes to form an emulsion. 1 g of calcium chloride solution (4.5%, pH 2.0) was slowly dropped to the emulsion and stirred for 30 minutes, allowing sodium alginate to crosslink to form calcitonin liposome polymeric microsphere (encapsulation efficiency (E.E.) was higher than 70.7%). The resultant microspheres were filtered off under reduced pressure. The filter cake was dispersed in water for 10 minutes (filter cake:water=1:3(w/w)), and then frozen at −20° C. for 3 hours. After complete freezing, the sample was freeze-dried for 24 hours, that is, frozen at −40° C. for 60 minutes and then dried at 4° C. until completely dry, obtaining dried polymeric microspheres.

### EXAMPLES 13-27

The procedures as described in Example 12 were again employed, except that some conditions were changed. The various conditions and results are shown in Table 4.

TABLE 4

| | Calcitonin | Sodium | | | | | |
|---|---|---|---|---|---|---|---|
| | liposome | alginate | Chitosan | Chitosan | | | |
| | conc. | conc. | 72 KDa | 180 KDa | CaCl₂ | ZnSo₄ | |
| Example | (mg/mL) | (%) | (%) | (%) | (%) | (%) | E.E. (%) |
| 13 | 0.25 | 5 | 1.5, | | 4.5, | | 90.0 |
| | | | pH 2.0 | | pH 2.0 | | |
| 14 | 0.5 | 5 | 1.5, | | | 4.5, | 93.8 |
| | | | pH 2.0 | | | pH 2.0 | |
| 15 | 0.67 | 3.3 | 1.5, | | 4.5, | | 71.0 |
| | | | pH 2.0 | | pH 2.0 | | |
| 16 | 0.67 | 3.3 | 2, | | 4.5, | | 84.9 |
| | | | pH 2.0 | | pH 2.0 | | |
| 17 | 0.33 | 3.3 | 2, | | 6, | | 74.1 |
| | | | pH 2.0 | | pH 2.0 | | |
| 18 | 0.33 | 3.3 | 2, | | 6, | | 83.2 |
| | | | pH 2.0 | | pH 2.0 | | |
| 19 | 0.33 | 3.3 | 2, | | | 6, | 94.5 |
| | | | pH 2.0 | | | pH 2.0 | |
| 20 | 0.33 | 3.3 | 1, | | | 6, | 88.5 |
| | | | pH 2.0 | | | pH 2.0 | |
| 21 | 0.37 | 2.5 | 2, | | | 6, | 59.9 |
| | | | pH 2.0 | | | pH 2.0 | |
| 22 | 0.37 | 2.5 | 1, | | | 6, | 55.5 |
| | | | pH 2.0 | | | pH 2.0 | |
| 23 | 0.4 | 2 | 2, | | | 6, | 62.0 |
| | | | pH 2.0 | | | pH 2.0 | |
| 24 | 0.4 | 2 | 1, | | | 6, | 59.8 |
| | | | pH 2.0 | | | pH 2.0 | |
| 25 | 0.37 | 2.5 | | 2, | | 6, | 91.8 |
| | | | | pH 2.0 | | pH 2.0 | |
| 26 | 0.37 | 2.5 | | 1, | | 6, | 89.8 |
| | | | | pH 2.0 | | pH 2.0 | |
| 27 | 0.4 | 2 | | 2, | | 6, | 65.9 |
| | | | | pH 2.0 | | pH 2.0 | |

### EXAMPLE 28

### Preparation of Liposome Encapsulated with Insulin

0.5 g lipid was added to a 15 mL tube. The lipid contained soybean phosphatidylcholine, polyoxyethylene (23) lauryl ether (Brij 35), cholesterol, and medium-chain triglyceride oil (3575oil) with a molar ratio of 10:1:2:1.16. Proper quantities of insulin were dissolved in 0.01M phosphate buffer solution (pH 7.4). 0.5 ml insulin solution (60 mg/mL) was then added to the lipid and mixed with vortex at 200 rpm for 1 hour to form a colloid. 0.2 g colloid and 1.8 mL 30 mM phosphate buffer solution (pH 7.4) were then added to a 10 mL flask and hydrated at room temperature for 1 hour. An insulin liposome solution was prepared. The encapsulation efficiency of the insulin liposome was 82.1%.

### EXAMPLES 29 and 30

The procedures as described in Example 28 were again employed, except that lipid formulations were changed. The results are shown in Table 5.

TABLE 5

| | | | | | | | | Osmosis | |
|---|---|---|---|---|---|---|---|---|---|
| | | | Brij | 3575 | PS | | E.E. | pressure | Hydration |
| Example | SPC | Chol. | 35 | oil | (nm) | PI | (%) | (mmol/kg) | solution |
| 28 | 10 | 2 | 1 | 1.16 | 226.6 | 0.093 | 82.1 | 25/856 | 30 mM PBS |
| 29 | 10 | 2 | 1 | 1.16 | 235.1 | 0.218 | 71.2 | 25/290 | 10 mM PBS |
| 30 | 10 | 1 | 1 | | 263.2 | 0.423 | 56.0 | 25/290 | 10 mM PBS |

In insulin liposome preparation, removal of drugs from inner buffer to hydration buffer is decreased during hydration due to formation of concentration gradient between liposome core and hydration buffer, increasing encapsulation efficiency.

### EXAMPLE 31

### Preparation of Liposome Encapsulated with Insulin

0.5 g lipid was added to a 15 mL tube. The lipid contained soybean phosphatidylcholine, α-tocopherol succinate PEG 1500 (TPGS), cholesterol, and 1,2-dipalmitoyl-sn-glycero-3-phosphatidylglycerol (DPPG) with a molar ratio of 10:1:1:1. Proper quantities of insulin were dissolved in 0.01M phosphate buffer solution (pH 7.4). 0.5 mL insulin solution (60 mg/mL) was then added to the lipid and mixed with vortex at 200 rpm for 1 hour to form a colloid. 0.2 g colloid and 0.8 mL 30 mM phosphate buffer solution (pH 7.4) were then added to a 10 mL flask and hydrated at room temperature for 1 hour. An insulin liposome solution was prepared. The encapsulation efficiency of the insulin liposome was 80.2%.

### EXAMPLE 32

The procedures as described in Example 31 were again employed, except that lipid formulations were changed. The results are shown in Table 6.

TABLE 6

| | | | | | P.S. | | |
|---|---|---|---|---|---|---|---|
| Example | SPC | Chol. | DPPG | TPGS | (nm) | P.I. | E.E. (%) |
| 31 | 10 | 1 | 1 | 1 | 274.8 | 0.068 | 80.2 |
| 32 | 10 | 1 | | 1 | 246.9 | 0.258 | 55.9 |

The DPPG (anionic lipid) significantly increases encapsulation efficiency to 80.2%.

### EXAMPLE 33

### Preparation of Liposome Encapsulated with Insulin

0.5 g lipid was added to a 15 mL tube. The lipid contained soybean phosphatidylcholine, polyoxyethylene (23) lauryl ether (Brij 35), cholesterol, and medium-chain triglyceride oil (3575oil) with a molar ratio of 10:2:1:1.16. Proper quantities of insulin were dissolved in 0.1M phosphate buffer solution (pH 7.4). 0.5 ml insulin solution (200 mg/mL) was then added to the lipid and mixed with vortex at 200 rpm for 1 hour to form a colloid. 0.2 g colloid and 0.8 mL 30 mM phosphate buffer solution (pH 7.4) were then added to a 10 ml flask and hydrated at room temperature for 1 hour. An insulin liposome solution was prepared. The encapsulation efficiency of the insulin liposome was 70.2% or more. The drug content thereof was 31.6 mg/mL.

### EXAMPLES 34-36

The procedures as described in Example 33 were again employed. The results are shown in Table 7.

TABLE 7

| | | | | | | | | | Initial |
|---|---|---|---|---|---|---|---|---|---|
| | | | | | | | | | insulin |
| | | | Brij | 3575 | P.S. | | E.E. | Total conc. | conc. |
| Example | SPC | Chol. | 35 | oil | (nm) | P.I. | (%) | (mg/mL) | (mg/mL) |
| 33 | 10 | 2 | 1 | 1.16 | 316.0 | 0.649 | 70.2 | 31.6 | 200 |
| 34 | 10 | 2 | 1 | 1.16 | 216.7 | 0.190 | 79.1 | 6.2 | 60 |
| 35 | 10 | 2 | 1 | 1.16 | 230.9 | 0.225 | 74.7 | 13.3 | 75 |
| 36 | 10 | 2 | 1 | 1.16 | 273.4 | 0.327 | 75.3 | 16.3 | 100 |

The encapsulation efficiency of liposomes is increased (>70%) by adding 3575oil.

### EXAMPLE 37

### Preparation of Polymeric Microsphere Encapsulated with Insulin Liposomes

10% sodium alginate solution, 1.5% chitosan solution, and 4.5% of calcium chloride solution were prepared and adjusted to pH 2.0. 0.33 mL of an insulin liposome solution and 0.67 g of 10% sodium alginate solution were mixed and then added to 2 mL of the chitosan solution. The resulting solution was homogenized at 9500 rpm for 1 minute to form an emulsion. 1 mL of 4.5% calcium chloride solution was added to the emulsion and stirred for 5 minutes, obtaining a polymer microsphere solution encapsulated with insulin. The resultant microspheres were filtered off under reduced pressure. The filter cake was dispersed in water for 10 minutes (filter cake:water=1:3(w/w)), and then frozen at −20° C. for 3 hours. After complete freezing, the sample was freeze-dried for 24 hours, that is, frozen at −40° C. for 60 minutes and then dried at 4° C. until completely dry, obtaining dried polymeric microsphere.

### EXAMPLES 38-46

The procedures as described in Example 37 were again employed, except that some conditions were changed. The various conditions and results are shown in Table 8.

TABLE 8

| | Insulin | | | | | |
|---|---|---|---|---|---|---|
| | Liposome | Sodium | | | | |
| Exam- | conc. | Alginate | CP 934P | Chitosan | CaCl₂ | E.E. |
| ple | (mg/mL) | (%) | (%) | (%) | (%) | (%) |
| 38 | 4.0 | 3.3 | | 1.5, | 4.5 | 88.9 |
| | | | | pH 2 | | |
| 39 | 4.0 | 3.3 | | 1.0, | 6, | 30.7 |
| | | | | pH 1 | pH 1 | |
| 40 | 4.0 | 3.3 | | 1.0, | 6, | 94.1 |
| | | | | pH 2 | pH 2 | |
| 41 | 4.0 | 3.3 | | 1.0, | 6, | 77.9 |
| | | | | pH 3 | pH 3 | |
| 42 | 4.0 | 3.3 | | 1.0, | 6, | 74.3 |
| | | | | pH 4 | pH 4 | |
| 43 | 4.0 | 3.3 | | 1.0, | 6, | 97.4 |
| | | | | pH 5 | pH 5 | |
| 44 | 4.0 | 3.3 | | 1.0, | 6, | 97.4 |
| | | | | pH 5.85 | pH 5.85 | |
| 45 | 4.0 | 1.7 | 1.7 | 1.5, | 4.5 | 87.6 |
| | | | | pH 2 | | |
| 46 | 4.0 | 1.1 | 2.2 | 1.5, | 4.5 | 97.1 |
| | | | | pH 2 | | |

### EXAMPLE 47

### Preparation of Polymeric Microspheres by Aqueous Two Phase Method in Six Repetitions

The procedures as described in Example 37 were again employed, except that the concentration of sodium alginate was changed. Six repetitions were performed. The results are shown in Table 9.

TABLE 9

| | Sodium | | | | Drug content |
|---|---|---|---|---|---|
| | Alginate | Chitosan | CaCl₂ | E.E. | (mg/g |
| Example | (%) | (%) | (%) | (%) | micropheres) |
| 47-1 | 3.3 | 1.5 pH 2 | 4.5 pH 2 | 88.9 | * |
| 47-2 | 3.3 | 1.5 pH 2 | 4.5 pH 2 | 84.1 | * |
| 47-3 | 3.3 | 1.5 pH 2 | 4.5 pH 2 | 87.3 | * |
| 47-4 | 3.3 | 1.5 pH 2 | 4.5 pH 2 | 82.5 | 39.7 |
| 47-5 | 3.3 | 1.5 pH 2 | 4.5 pH 2 | 86.9 | 37.6 |
| 47-6 | 3.3 | 1.5 pH 2 | 4.5 pH 2 | 87.4 | 38.2 |

It can be seen from Table 9 that the aqueous-two-phase method for preparing sodium alginate polymeric microspheres exhibits good repeatability. In addition, the encapsulation efficiency (E.E.) of insulin liposome is as high as 86.2%, and the CV (coefficient of variation) is 2.77%.

### COMPARATIVE EXAMPLES 48 and 49

The procedures as described in Example 38 were again employed, except that the spray nozzle method was used. The obtained polymeric microspheres encapsulated with insulin liposomes were 0.1 g. Table 10 shows a comparison between the results of Example 38, comparative examples 48 and 49.

TABLE 10

| | | Particle | | | | | |
|---|---|---|---|---|---|---|---|
| | | Size of | | Drug | | | |
| | Polymer | Polymer | | Content | | | |
| | Micropheres | Micropheres | E.E. | (mg/g | Recovery | | |
| Example | (g) | (μm) | (%) | microphere) | (%) | Method | Apparatus |
| Comp. Exp. | 0.1 | 27.37 | 93.7 | 20.7 | 76.4 | Spray | 0.54 mm |
| 48 | | | | | | Nozzle | Nozzle |
| Comp. Exp. | 0.1 | 15.08 | 85.4 | 21.3 | 76.4 | Spray | 0.54 mm |
| 49 | | | | | | Nozzle | Nozzle |
| Example 38 | 0.1 | 2.51 | 88.9 | 37.8 | 90.1 | Aqueous- | Probe type |
| | | | | | | two-phase | homogenizer |
| | | | | | | Emulsion | |

It can be seen from Table 10 that the aqueous-two-phase method of the present invention provides polymeric microsphere with a high recovery of 90%. Nevertheless, using the conventional spray nozzle method to prepare polymeric microsphere only obtains a recovery yield of 74-76%.

### EXAMPLE 50

The procedures as described in Example 38 were again employed, except that insulin liposome was not encapsulated and the reactant amounts were scaled up such that the obtained sodium alginate polymeric microsphere was 5 g.

Table 11 shows the result of triplet repetitions.

TABLE 11

| | | Particle | | | |
|---|---|---|---|---|---|
| | Polymer | Size of | Rate of | Homoge- | Cross- |
| | Micro- | Polymer | Homoge- | nization | linking |
| | spheres | Microspheres | nizer | Time | Time |
| Example | (g) | (μm) | (rpm) | (min) | (min) |
| 50-1 | 5 | 2.09 | 3000 | 1 | 5 |
| 50-2 | 5 | 2.09 | 5000 | 1 | 5 |
| 50-3 | 5 | 2.12 | 3000 | 5 | 5 |

This example enlarges the aqueous-two-phase process to prepare 5 g of polymeric microsphere using a homogenizer at 3000-5000 rpm for 1-5 minutes. The obtained sodium alginate polymeric microspheres had a relative uniform particle size, on an average, of 2.10 μm, and CV(%) was 0.85%.

### EXAMPLE 51

400 g 10% sodium alginate solution, 2000 mL 1.5% chitosan solution, and 1000 mL 4.5% calcium chloride solution were prepared. Then, the chitosan and calcium chloride solutions were adjusted to pH 2. 400 g of 10% sodium alginate solution and 800 g of an insulin liposome solution were mixed to form 1200 g of a mixed solution. After complete mixing, 1000 g of the sodium alginate/insulin liposome solution was added to 2000 mL of chitosan solution and then homogenized by a continuous homogenizer and a 5 Liter circulation tube at 21000 rpm for 60 minutes to form an emulsion. 1000 mL of the calcium chloride solution was then added slowly and the mixture was stirred at 250 rpm for 30 minutes in order to cross-link sodium alginate to form insulin liposome microspheres. The reaction solution was poured in a 4 Liter plate-type filter press in two batches and filter pressed at 3 kg/cm² for separation. The obtained filter cake was dispersed in water (filter cake:water=1:3(w/w)), then poured in a 35 cm×25 cm stainless steel plate (the liquid height was not higher than 0.5 cm), and then frozen at −20° C. for 3 hours. After complete freezing, the sample was freeze-dried for 24 hours, that is, frozen at −40° C. for 60 minutes and then dried at 4° C. until completely dry, obtaining 100 g of dried insulin polymeric microsphere. The encapsulation efficiency reached up to 87.8% and the recovery reached up to 94.8%.

### EXAMPLES 52-54

The procedures as described in Example 51 were again employed, except that the reactant amounts were changed such that the insulin liposome-encapsulated sodium alginate microsphere amounts obtained were different. The results are shown in Table 12.

### EXAMPLES 55 and 56

The procedures as described in Example 51 were again employed, except that the reactant amounts were changed such that the insulin liposome-encapsulated sodium alginate microsphere amounts obtained were different, and that a continuous homogenization method was used. The results are shown in Table 12.

TABLE 12

| | | Particle | | | | | |
|---|---|---|---|---|---|---|---|
| | | Size of | | Drug | | | |
| | Polymer | Polymer | | Content | | | |
| | Micropheres | Micropheres | E.E. | (mg/g | Recovery | | |
| Example | (g) | (μm) | (%) | microphere) | (%) | Method | Apparatus |
| 52 | 0.1 | 2.51 | 88.9 | 37.8 | 90.7 | Batch | Probe type |
| | | | | | | | homogenizer |
| 53 | 5 | 2.59 | 90.1 | 39.4 | 91.3 | Batch | Probe type |
| | | | | | | | Homogenizer |
| 54 | 10 | 2.49 | 88.5 | 38.9 | 89.4 | Batch | Probe type |
| | | | | | | | Homogenizer |
| 55 | 50 | 2.29 | 90.2 | 38.5 | 94.0 | Continuous | Continuous |
| | | | | | | | type |
| | | | | | | | Homogenizer |
| 56 | 100 | 3.27 | 89.4 | 38.0 | 94.7 | Continuous | Continuous |
| | | | | | | | type |
| | | | | | | | Homogenizer |

It can be seen from Table 12 that using the aqueous-two-phase method of the present invention generates 4 liters of emulsion and 100 g of dried polymeric microspheres. Also, the polymeric microspheres had good encapsulation efficiency and their drug contents had good repeatability. In addition, when the process was made continuous, the recovery was increased from 90% to 94%.

In conclusion, the invention uses two miscible polymer solutions to perform polymeric microspheres. The present invention does not require any organic solvent or surfactant. Therefore, the encapsulated biological drug is not deactivated. The recovery and the encapsulation efficiency of the drug are high.

While the invention has been described by way of examples and in terms of preferred embodiment, it is to be understood that the invention is not limited thereto. To the contrary, it is intended to cover various modifications and similar arrangements (as would be apparent to those skilled in the art). Therefore, the scope of the appended claims should be accorded the broadest interpretation so as to encompass all such modifications and similar arrangements.

What is claimed is:

1. A polymeric microsphere, comprising:
a first polymer;
a layer formed on the surface of the first polymer; and
a second polymer formed on the layer.

2. The polymeric microsphere as claimed in claim 1, wherein the first polymer comprises carboxylate (COO⁻) or carboxyl (COOH) groups.

3. The polymeric microsphere as claimed in claim 1, wherein the first polymer comprises alginic acid, alginate, propylene glycol alginate, carboxylmethyl cellulose, polyacrylic acid, or polyacrylate derivatives.

4. The polymeric microsphere as claimed in claim 3, wherein the alginate comprises sodium alginate.

5. The polymeric microsphere as claimed in claim 1, wherein the first polymer and second polymer are miscible.

6. The polymeric microsphere as claimed in claim 1, wherein the second polymer comprises chitosan, starch, dextran, hydroxyl propyl methyl cellulose, or gelatin.

7. The polymeric microsphere as claimed in claim 2, wherein the layer is formed by cross-linking of the carboxylate (COO⁻) or carboxyl (COOH) groups on the surface of the first polymer.

8. The polymeric microsphere as claimed in claim 1, wherein the microsphere encapsulates a plurality of liposomes.

9. The polymeric microsphere as claimed in claim 8, wherein the liposomes encapsulate drugs.

10. The polymeric microsphere as claimed in claim 1, wherein the microsphere encapsulates drugs.

11. A method for preparing polymeric microspheres, comprising:
providing a first polymer aqueous solution, wherein the first polymer having functional groups capable of cross-linking;
providing a second polymer aqueous solution, wherein the first and second polymer aqueous solutions are miscible;
mixing the first and second polymer aqueous solutions to form a mixture; and
adding a crosslinking agent to form a microsphere.

12. The method as claimed in claim 11, wherein the first polymer comprises carboxylate (COO⁻) or carboxyl (COOH) groups.

13. The method as claimed in claim 11, wherein the first polymer comprises alginic acid, alginate, propylene glycol alginate, carboxylmethyl cellulose, polyacrylic acid, or polyacrylate derivatives.

14. The method as claimed in claim 13, wherein the alginate comprises sodium alginate.

15. The method as claimed in claim 11, wherein the second polymer comprises chitosan, starch, dextran, hydroxyl propyl methyl cellulose, or gelatin.

16. The method as claimed in claim 11, wherein the second polymer aqueous solution has pH of 0.5 to 6.

17. A method for preparing polymeric microspheres, comprising:
providing a first polymer aqueous solution, wherein the first polymer having functional groups capable of cross-linking;
providing a second polymer aqueous solution, wherein the first and second polymer aqueous solutions are miscible;
mixing drugs and the first polymer aqueous solution to form a drug aqueous solution;
mixing the drug aqueous solution and the second polymer aqueous solution to form a mixture; and
adding a crosslinking agent to form a microsphere.

18. The method as claimed in claim 17, wherein the first polymer comprises carboxylate (COO⁻) or carboxyl (COOH) groups.

19. The method as claimed in claim 17, wherein the first polymer comprises alginic acid, alginate, propylene glycol alginate, carboxylmethyl cellulose, polyacrylic acid, or polyacrylate derivatives.

20. The method as claimed in claim 19, wherein the alginate comprises sodium alginate.

21. The method as claimed in claim 17, wherein the second polymer comprises chitosan, starch, dextran, hydroxyl propyl methyl cellulose, or gelatin.

22. The method as claimed in claim 17, wherein the second polymer aqueous solution has a pH of 0.5 to 6.

23. A method for preparing polymeric microspheres, comprising:
providing a first polymer aqueous solution, wherein the first polymer having functional groups capable of cross-linking;
providing a second polymer aqueous solution, wherein the first and second polymer aqueous solutions are miscible;
mixing a liposome solution and the first polymer aqueous solution to form a solution;
mixing the solution and the second polymer aqueous solution to form a mixture; and
adding a crosslinking agent to form a microsphere.

24. The method as claimed in claim 23, wherein the first polymer comprises carboxylate (COO⁻) or carboxyl (COOH) groups.

25. The method as claimed in claim 23, wherein the first polymer comprises alginic acid, alginate, propylene glycol alginate, carboxylmethyl cellulose, polyacrylic acid, or polyacrylate derivatives.

26. The method as claimed in claim 25, wherein the alginate comprises sodium alginate.

27. The method as claimed in claim 23, wherein the second polymer comprises chitosan, starch, dextran, hydroxyl propyl methyl cellulose, or gelatin.

28. The method as claimed in claim 23, wherein the second polymer aqueous solution has a pH of 0.5 to 6.

29. The method as claimed in claim 23, wherein the liposomes encapsulate drugs.
